(19) United States
(12) Patent Application Publication
(10) Pub. No.: US 2022/0096340 A1
(43) Pub. Date: Mar. 31, 2022

(54) CURLING MASCARA AND METHOD OF USE

(71) Applicant: L'ORÉAL, Paris (FR)

(72) Inventors: Christopher PANG, Nanuet, NY (US); Sylvie FORET-FRISTOT, Clark, NJ (US); Chunhua LI, Hillsborough, NJ (US)

(73) Assignee: L'ORÉAL, Paris (FR)

(21) Appl. No.: 17/038,180

(22) Filed: Sep. 30, 2020

(51) Int. Cl. A61K 8/19; A41G 5/02; A61Q 1/10; A45D 2/48; A61K 8/81

(52) U.S. Cl. CPC A61K 8/19; A41G 5/02; A61Q 1/10; A61K 2800/87; A61K 8/8176; A61K 2800/30; A45D 2/48

(57) ABSTRACT

A method and mascara capable of providing semi-permanent curl to eyelashes is provided. The method involves applying a mascara that is substantially free of waxes and oxidizers to an eyelash, such as a magnetic eyelash. The mascara contains greater than 10% and less than 50% by weight of at least one ferromagnetic pigment, and between 9% and 18% solids content of a contractile polymer. After application, a magnet is temporarily held or moved over the eyelash in the direction of curl. Preferably the curling is accomplished without using a brush or eyelash tool.

### TECHNICAL FIELD

The present invention relates to compositions and techniques for curling eyelashes, and specifically to mascara compositions that create curl which can be further enhanced through the use of magnets.

### BACKGROUND

Mascaras are traditionally used to improve the perceived aesthetics of a person's eyelashes. Such aesthetic qualities include the degree of curl. Conventional Mascara utilize waxes, surfactant, and film formers to improve the curl of the eyelashes. However, such techniques are inherently limited by available chemistries and the biological properties of a given person's eyelashes.

Therefore, a technique for enhancing the curling eyelashes, that avoids some of the inherent biological or chemical limitations, is desirable.

### BRIEF SUMMARY

A first aspect of the present disclosure is drawn to a technique for providing semi-permanent curl to eyelashes. The method involves applying (via, e.g., an applicator) a mascara containing a ferromagnetic pigment and a contractile polymer to a natural or synthetic eyelash (e.g., such as a magnetic eyelash). The mascara is typically applied to a single side of the eyelash, such as the underside of the eyelash (the side of the eyelash that closest to the eye). Upon application, the contractile polymer will begin contracting, causing the eyelash to curl.

After application, a magnet can be temporarily held in place over the eyelash, or moved around over the eyelash, in the direction curl is desired. The magnet attracts the ferromagnetic pigment, synergistically enhancing the curling efforts being undertaken by the contractile polymer.

This method can be accomplished with or without use of an eyelash tool or brush.

A second aspect of the present disclosure is drawn to mascara compositions for use on eyelashes and enhanced via use of a magnet. The mascara utilizes greater than 10% and less than 40% by weight of at least one ferromagnetic pigment; and between 9% and 18% solids content of a contractile polymer. The mascara is substantially free of waxes and oxidizers.

Optionally, the mascara comprises between 15% and 35% by weight of the at least one ferromagnetic pigment. Optionally, the at least one ferromagnetic pigment is uncoated.

Optionally, the mascara comprises between 40% and 85% by weight of the contractile polymer. Optionally, the contractile polymer has a glass transition temperature Tg≥45° C. Optionally, the mascara is substantially free of a C1-C4 monoalcohol. Optionally, the mascara is substantially free of water.

In some embodiments, the mascara also contains at least one non-ferromagnetic pigment, and/or at least one film forming polymer in addition to the contractile polymer.

In some embodiments, the contractile polymer is a film-forming polymer comprising at least one cyclic group selected from the group consisting of cyclic amides, cyclic amines, and mixtures thereof, and optionally, the cyclic amide group and/or cyclic amine group of the at least one film forming polymer comprise one or more aromatic or aliphatic ring structures.

In some embodiments, the contractile polymer is a copolymer comprising at least one pyrrolidone monomer. Optionally, less than 60% of the monomers in the copolymer are pyrrolidone monomers.

In some embodiments, the contractile polymer has at least some acrylate or acrylamide functionality.

### BRIEF DESCRIPTION OF THE DRAWINGS

FIG. 1 is an illustration of an embodiment of a system according to the present invention, including an eyelash, eyelid, and magnet.

### DETAILED DESCRIPTION

As used herein, articles such as “a” and “an” when used in a claim, are understood to mean one or more of what is claimed or described.

As used herein, the term “about [a number]” is intended to include values rounded to the appropriate significant digit. Thus, “about 1” would be intended to include values between 0.5 and 1.5, whereas “about 1.0” would be intended to include values between 0.95 and 1.05.

As used herein, the term “at least one” means one or more and thus includes individual components as well as mixtures/combinations.

As used herein, the term “free [of an ingredient]” means that the identified ingredient is only present in an amount below its detectable limit, and preferably that the composition contains 0% of the identified ingredient.

As used herein, the terms “include”, “includes” and “including” are meant to be non-limiting.

As used herein, the term “substantially free [of an ingredient]” means that the composition contains less than 1% of the identified ingredient.

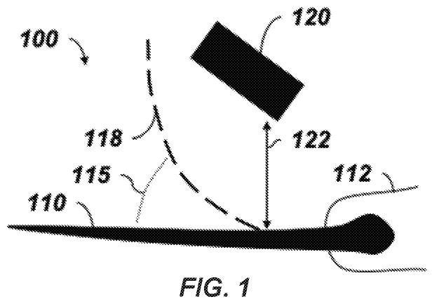

Disclosed is a method for providing semi-permanent curl to eyelashes, which can best be understood with reference to FIG. 1. In the disclosed system 100, an eyelash 110 is first provided. This may be a natural or synthetic eyelash, such as a magnetic eyelash. The eyelash 110 in FIG. 1 is a natural eyelash, and the eyelash 110 is shown extending from an eyelid 112.

A mascara is then applied to the eyelash 110. This may be done using an applicator (not shown) or any other appropriate technique for introducing the mascara to the eyelash 110. The mascara is typically applied to a single side of the eyelash, such as the underside of the eyelash (the side of the eyelash that closest to the eye), although it can be applied to other portions of the eyelash as well. In reference to FIG. 1, the mascara is preferably applied to the surface of eyelash 110 that faces away from magnet 120. The application process typically bends the eyelash at least somewhat in the direction curl is desired (e.g., curling the lash away from the eye).

The mascara comprises a ferromagnetic pigment and a contractile polymer.

Ferromagnetic Pigment

The mascara may include one or more ferromagnetic pigments. As used herein, the term “ferromagnetic pigment” means a cosmetically acceptable material that exhibits ferromagnetic properties, including being attracted by a magnet. These materials include metals and metal oxides, such as iron oxide. A particularly suitable pigment is black iron oxide (CI 77499) which is a ferrous-ferric oxide. The pigment may be pearlescent. The pigment may be coated or uncoated, and are preferably uncoated. The pigment may be chemically or physically coupled to a supporting material (e.g., a polymer, silica, mica, etc.) in order to provide the desired aesthetic effect (such as a matte, pearled, satin, or metallic effect).

The ferromagnetic pigments comprise at least 50%, at least 60%, at least 70%, at least 80%, at least 90%, or 100% of all colorants in the mascara.

In some embodiments, the ferromagnetic pigments are present in a total amount >10% by weight and <50% by weight in the mascara. In some embodiments, the ferromagnetic pigments are present in a total amount >10% by weight and <40% by weight in the mascara. In some embodiments, the ferromagnetic pigments are present in a total amount >10%, ≥10%, ≥15%, ≥20%, and present in a total amount ≤50%, ≤45%, ≤40, or ≤35 and or any combination thereof. For example, in one embodiment, the mascara comprises between 15% and 35% by weight of the at least one ferromagnetic pigment.

Contractile Polymer

The mascara may include one or more contractile polymers. A contractile polymer is a polymer contracts and hardens when it dries. Some film-forming polymers are contractile polymers.

The glass transition temperature of the contractile polymer should be greater than or equal to 45° C. In some embodiments, Tg is ≥80° C., ≥117° C., ≥125° C., and/or ≥200° C. The Tg is preferably less than 400° C.

As C1-C4 monoalcohols (such as ethanol) can interfere with the film forming process, the second composition should avoid the use of such C1-C4 monoalcohols, and ethanol in particular. Disclosed compositions are therefore preferably free, or substantially free, of C1-C4 monoalcohols. Other alcohols, such as glycols, are preferably only present at low levels (<5% by weight relative to the contractile polymer)

Preferred copolymers that can function as contractile polymers do not contain a substantial majority of monomers that are water soluble, and preferably less than 60% of the monomers are water soluble. For copolymers that contains a pyrrolidone monomer (such as N-vinylpyrrolidone), less than 60% of the monomers should be the pyrrolidone monomer, and preferably less than about 50%.

Preferably, the contractile polymer contains at least some acrylate or acrylamide functionality. For example, copolymers that include methyacrylamide, acrylate copolymer, styrene/acrylates copolymers, and polyacrylates such as polyacrylate-22.

The contractile polymer may be one or more film-forming polymers comprising at least one cyclic group selected from the group consisting of cyclic amides, cyclic amines, and mixtures thereof. Preferably, the at least one film-forming polymer comprising at least one cyclic group selected from the group consisting of cyclic amides, cyclic amines, and mixtures thereof is non-ionic and water-soluble or water-dispersible. Without wishing to be bound by theory, it is believed that the presence of a sufficient amount of the at least one film forming polymer comprising at least one cyclic group selected from the group consisting of cyclic amides, cyclic amines, and mixtures thereof in the compositions of the present invention results in a curling effect on hair, eyebrows and/or eyelashes after application to the hair, eyebrows and/or eyelashes (and after the applied composition has dried).

According to preferred embodiments, the film forming polymer comprising at least one cyclic group selected from the group consisting of cyclic amides, cyclic amines, and mixtures thereof has a weight average molecular weight in a range from about 10,000 daltons to about 1,000,000 daltons, preferably from about 20,000 daltons to about 800,000 daltons, preferably from about 50,000 daltons to about 600,000 daltons, and preferably from about 100,000 daltons to about 500,000 daltons, including all ranges and subranges therebetween such as, for example, 15,000 daltons to 900,000 daltons, 200,000 daltons to 400,000 daltons, 10,000 daltons to 150,000 daltons, etc.

Preferably, the cyclic amide group and/or cyclic amine group of the at least one film forming polymer comprise one or more aromatic or aliphatic ring structures. Preferably, the rings have a size of from 4 to 10 ring members, preferably 5 to 8 ring members, and preferably 5 to 6 ring members, including all ranges and subranges therebetween.

Preferably, the cyclic amide group and/or cyclic amine group of the at least one film forming polymer are polymerizable ethylenically unsaturated monomers having a cyclic amine residue or a cyclic amide residue. Accordingly, the cyclic amide groups or monomers of the film-forming polymers useful in the present invention may include cyclic amide residues that are, or include, heterocyclic ring structures such as lactams and the like such as, for example, α-Lactam, β-lactam, γ-lactam, δ-lactam, and ε-lactam. Preferably, the cyclic amide is a pyrrolidone (a γ-lactam) a caprolactam, or combinations thereof.

Preferred cyclic amine groups include various heterocyclic amines such as, for example, azoles, pyrroles, pyrrolidines, carbamates, and the like. Preferably, the cyclic amine group is an imidazole.

Optionally, the contractile polymers may comprise other groups in addition to the cyclic amide groups and/or cyclic amine groups.

If present, the additional group(s) are preferably acrylamide monomer(s), preferably having one or more —C3H5NO functional groups. Specific examples of such additional groups include, but are not limited to, (meth)acrylamides.

An example of a preferred contractile polymer is LUVISET® CLEAR AT3, a copolymer of N-vinyl pyrrolidone, methacrylamide, and N-vinylimidazole commercially available from BASF of Ludwigshafen, Germany.

Preferably, the contractile polymer is present in an amount such that there is between 9% and 18% solids content of the contractile polymer in the mascara. Preferably, the maximum solids content of the contractile polymer in the mascara is ≤18%, ≤17%, ≤16%, or ≤15%, and the minimum solids content of the contractile polymer is ≥9%, ≥10%, or ≥11%, or any combination thereof.

In some embodiments, the ratio of solids content of the contractile polymer to the amount of ferromagnetic pigment, by wt %, present in the mascara is ≥0.18:1, ≥0.186:1, ≥0.2:1, or ≥0.5:1 and is ≤1.8:1, ≤1.7:1, ≤1.67:1, or ≤1.5:1, or any combination thereof.

Other Materials

Although the disclosed compositions are free, or substantially free, of waxes and oxidizers, the mascara may comprise or consist of the contractile polymers, the ferromagnetic pigments, and other components, including, e.g., solvents, preservatives, additional film former agents, and/or colorants other than ferromagnetic pigments. In some embodiments, the mascara is free, or substantially free, of water.

Additional Film Former

Optionally, embodiments of the second composition may further comprise at least one additional film forming polymer in addition to the contractile polymer. If present, the at least one additional film forming polymer may be any film forming polymer suitable for use in a composition for application to hair, eyebrows and/or eyelashes.

For example, the at least one additional film forming polymer may include at least one cyclic amide monomer (hereinafter “second cyclic amide monomer”). The fraction (e.g., weight fraction) of the second cyclic amide monomer in the at least one additional film forming polymer is at least about 70%, preferably at least about 75%, and preferably at least about 80%. Further, the second cyclic amide monomer is preferably selected from a vinyl pyrrolidone (a γ-lactam) a caprolactam, and combinations thereof.

Suitable examples of the additional film forming polymer include, but are not limited to, vinyl pyrrolidone/vinyl acetate copolymers having at least 70% vinyl pyrrolidone monomer, such as LUVIKSOL 73E, LUVIKSOL 73W; polyvinylcaprolactam, such as LUVIKSOL Plus; and polyvinyl pyrrolidone homopolymer such as PVP K-60 (or PLASDONE K-60), PVPK-90 (or PLASDONE K-90), or PVP K-120 (or PLASDONE K-120), each commercially available from Ashland, Inc. of Kovington, Ky.

Specific examples also include, but are not limited to, a silicone polymer such as, for example, a non-ionic silicone copolymer such as a non-ionic dimethicone copolymer. The silicone polymer or copolymer may be in the form of particles dispersed in an aqueous dispersion medium. Non-limiting examples of non-ionic silicone polymers include polymethylsiloxane resin, a linear block copolymer, and a mixture thereof. More specifically, non-limiting examples include a dimethicone copolymer such as a copolymer of dimethylpolysiloxane and vinyl dimethylpolysiloxane (i.e., a polydimethylsiloxane/vinyl copolymer) or a copolymer of dimethylpolysiloxane and a (meth)acrylate, with the dimethicone copolymer optionally being crosslinked and/or end-capped with functional groups. For example, a polydimethylsiloxane and vinyl dimethylpolysiloxane may comprise dimethylpolysiloxane that is crosslinked with vinyl dimethylpolysiloxane and/or dimethylpolysiloxane that is end-capped with vinyl dimethylpolysiloxane. A preferred compound includes dimethylpolysiloxane crosslinked with vinyl dimethylpolysiloxane. An example of a particularly useful dimethicone copolymer is a divinyl-dimethicone/dimethicone copolymer available as DOWSIL UMW 2220 Non-Ionic Emulsion, available from Dow Corning of Midland, Mich. This is a 60 percent active aqueous dispersion of divinyldimethicone/dimethicone copolymer and comprising C₁₂-C₁₃ Pareth-3 and C₁₂-C₁₃ Pareth-23.

Specific examples further include non-crosslinked acrylate and acrylic co-polymers, urethane polymers, polyesters and combinations thereof. A non-limiting example of a suitable non-crosslinked additional film forming agent is sodium alginate, available as PROTANAL PH 6160 from FMC Health and Nutrition of Philadelphia, Pa.

Preferably, if present, the additional film forming polymer is present in an amount ranging from about 0.1% to about 40% by weight, preferably from about 0.5% to about 30% by weight, preferably from about 1% to about 20% by weight, and preferably from about 2% to about 10% by weight, all weights being based on the total weight of the composition, including all ranges and subranges therebetween. Preferably, the compositions of the present invention contain 2% or less by weight of the additional film forming agent, preferably 1% or less by weight, preferably 0.5% or less by weight, all weights being based on the weight of the composition.

Preferably, the contractile polymer constitutes at least 55% percent by weight of the total film forming component of the mascara (i.e., the contractile polymer(s)+the additional film forming polymer(s)), at least 60% by weight, at least 75% by weight, at least 80% by weight, and least 90% by weight. Said differently, the ratio of contractile polymer(s) to additional film forming polymers should be >1.22 (55 contractile/45 additional), >1.5:1 (60/40), >3:1 (75/25), >4:1 (80/20), and >9:1 (90/20).

Other Colorants

Optionally, the disclosed compositions may include one or more other colorants.

Colorants are preferably chosen from pulverulent materials, liposoluble dyes and water-soluble dyes, and mixtures thereof.

Preferably, the compositions according to the invention comprise at least one pulverulent colorant. The pulverulent colorants may be chosen from pigments and nacres, and preferably from pigments.

The pigments may be white or colored, inorganic and/or organic, and coated or uncoated. Among the inorganic pigments, mention may be made of metal oxides, in particular titanium dioxide, optionally surface-treated, zirconium, zinc or cerium oxide, and also titanium or chromium oxide, manganese violet, ultramarine blue, chromium hydrate and ferric blue. Among the organic pigments that may be mentioned are carbon black, pigments of D&C type and lakes based on cochineal carmine or on barium, strontium, calcium or aluminum.

The nacres may be chosen from white nacreous pigments such as mica coated with titanium or with bismuth oxychloride, colored nacreous pigments such as titanium mica with iron oxides, titanium mica with in particular ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

In some embodiments, the mascara comprises at least one non-ferromagnetic pigment.

Referring back to FIG. 1 and the method, once the mascara has been applied, the contractile polymer will begin contracting, causing the eyelash to curl. As seen in FIG. 1, the eyelash will begin to curl from its normal, uncurled position 110, curling upwards towards its final curled position 118, curling at a deviation angle 115 that can be observed and measured visually.

After application, a magnet 120 can be temporarily held in place over the eyelash, or moved around over the eyelash 110, in the direction curl is desired, while the contractile polymer is fixing in place (or setting). The magnet attracts the ferromagnetic pigment, synergistically enhancing the curling efforts being undertaken by the contractile polymer. The magnet is preferably kept at a distance 122 from the eyelash, sufficient to ensure the magnetic field (not shown in FIG. 1) from the magnet 120 can interact with the ferromagnetic particles that have been applied to the eyelash. The actual distance will therefore be determined, in part, by the strength of the magnet utilized. Preferably, the minimum distance 122 of the magnet to the eyelash is >0 mm, ≥about 2.5 mm, ≥about 5 mm, ≥about 7.5 mm, ≥about 10 mm, ≥about 12.5 mm, or ≥about 15 mm, and preferably the minimum distance is ≤about 50 mm, ≤about 45 mm, ≤about 40 mm, ≤about 35 mm, ≤about 30 mm, ≤about 25 mm, ≤about 20 mm, ≤15 mm, or ≤about 12.5 mm, or any combination thereof.

In some embodiments, the eyelashes are exposed to the magnetic field from the magnet within 30 seconds, and preferably within 10 seconds, after the mascara has been applied to the eyelash. In some embodiments, the eyelashes are exposed to the magnetic field from the magnet for a total time of between 5 seconds and 1 minute. In preferred embodiments, the eyelash is exposed to the magnetic field for at least 5 seconds, at least 10 seconds, at least 15 seconds, at least 20 seconds, at least 25 seconds, or at least 30 seconds, and no more than 60 seconds, no more than 50 seconds, no more than 40 seconds, or no more than 30 seconds.

While any strength magnet is envisioned, some embodiments utilize magnets with a gauss rating of between 100 and 20,000.

### Example

A base mascara composition was created, to which varying amounts of pigment and/or contractile polymer were added. See Table 1, below. Each mascara composition was tested using artificial eyelashes, held in place with a thin flat clamp. The mascaras were applied with an applicator, and some of those test eyelashes were then exposed to a magnetic field from a hand-held magnet which was moved over the eyelashes 20 times, for a total exposure time of about 1 minute.

TABLE 1
Sample Mascaras

| Material | % w/w |
|---|---|
| Contractile Polymer (20% solids content) | 34-85% |
| Ferromagnetic Pigment | 10-60% |
| Preservative | 0-1% |
| Solvent | q.s. |

Evaluations

After the above method was used, the eyelashes were evaluated at their initial lift angles, and then after being subjected to hot and humid conditions (60% RH, 32° C.), for 20 minutes and evaluated again. The evaluations were done by holding each clamp horizontally, and measuring the angles formed by the eyelashes with a protractor. The viscosities of each composition with both pigment and contractile polymer was also measured. The results are summarized in Table 2, where a dash indicates the measurement was not taken.

TABLE 2
Summary of Evaluations

| % | % Solid Content | Curl | | Viscosity |
|---|---|---|---|---|
| Pigment | Contractile Polymer | No Magnet | With Magnet | (Pa s) |
| 10% | 0% | 0 | 0 | — |
| 0% | 16.7% | — | 0-20 | — |
| 10% | 16.7% | 30-50 | 10-50 | 3.96 |
| 20% | 0% | 0 | 0 | — |
| 0% | 14.88% | — | 10-30 | — |
| 20% | 14.88% | 0-30 | 10-60 | 2.53 |
| 30% | 0% | 0 | 0 | — |
| 0% | 13.02% | — | 10-30 | — |
| 30% | 13.02% | 0-20 | 20-50 | 2.74 |
| 40% | 0% | 0 | 0 | — |
| 0% | 11.16% | — | 0-15 | — |
| 40% | 11.16% | 20-40 | 10-45 | 13.3 |
| 50% | 0% | 0 | 0 | — |
| 0% | 9.3% | — | 0-15 | — |
| 50% | 9.3% | 0-10 | 10-30 | 10.7 |
| 60% | 0% | 0-10 | 0-10 | — |
| 0% | 6.9% | — | 0 | — |
| 60% | 6.9% | 0 | 0 | — |

As can be seen, the use of ferromagnetic pigments without a contractile polymer produce little to no curl, even when a magnet was used. The use of a contractile polymer without the ferromagnetic pigment provided some curl for use levels up at solid content levels more than about 9%. The combined use of the contractile polymer and the ferromagnetic pigments, in the presence of the magnet, saw a synergistic effect; for example, at 30% ferromagnetic pigment (0 curl on its own with a magnet) and about 13% solids content (10-30 degrees of curl on its own in the presence of a magnet) surprisingly saw 20-50 degrees of curl when a magnet was used while the contractile polymers set. This synergistic enhancement also surprisingly plummeted when the solids content was less than 9% and the ferromagnetic pigment concentration is above 50%.

Those skilled in the art will recognize or be able to ascertain using no more than routine experimentation many equivalents to the specific embodiments of the invention described herein. Such equivalents are intended to be encompassed by the following claims.

What is claimed is:

1. A method for providing semi-permanent curl to eyelashes, comprising:
providing a magnetic eyelash;
applying a mascara containing a ferromagnetic pigment and a contractile polymer to the magnetic eyelash; and
temporarily holding or moving a magnet over the magnetic eyelash in the direction of curl.

2. The method according to claim 1, further comprising allowing the magnetic eyelash to curl without using a brush, or eyelash tool.

3. A mascara for use on lashes containing a magnet, the mascara comprising:
greater than 10% and less than 50% by weight of at least one ferromagnetic pigment; and
between 9% and 18% solids content of a contractile polymer;
wherein the mascara is substantially free of waxes and oxidizers.

4. The mascara according to claim 3, wherein the mascara comprises between 15% and 35% by weight of the at least one ferromagnetic pigment.

5. The mascara according to claim 3, wherein the at least one ferromagnetic pigment is uncoated.

6. The mascara according to claim 3, wherein the contractile polymer has a glass transition temperature T_{g}≥45° C.

7. The mascara according to claim 3, wherein the mascara is substantially free of a C₁-C₄ monoalcohol.

8. The mascara according to claim 3, wherein the mascara is substantially free of water.

9. The mascara according to claim 3, further comprising at least one non-ferromagnetic pigment.

10. The mascara according to claim 3, wherein the mascara further comprises at least one film forming polymer in addition to the contractile polymer.

11. The mascara according to claim 3, wherein the contractile polymer is a film-forming polymer comprising at least one cyclic group selected from the group consisting of cyclic amides, cyclic amines, and mixtures thereof.

12. The mascara according to claim 11, wherein the cyclic amide group and/or cyclic amine group of the at least one film forming polymer comprise one or more aromatic or aliphatic ring structures.

13. The mascara according to claim 1, wherein the contractile polymer is a copolymer comprising at least one pyrrolidone monomer.

14. The mascara according to claim 13, wherein less than 60% of the monomers in the copolymer are pyrrolidone monomers.

15. The mascara according to claim 13, wherein the contractile polymer has at least some acrylate or acrylamide functionality.
